(19) United States
(12) Patent Application Publication
(10) Pub. No.: US 2009/0326595 A1
(43) Pub. Date: Dec. 31, 2009

(54) Prediction and Prevention of Cardiovascular Insult

(75) Inventors: Marina V. Brockway, Shoreview, MN (US); Brian P. Brockway, Shoreview, MN (US)

Correspondence Address: FISH & RICHARDSON P.C., P.O. BOX 1022, MINNEAPOLIS, MN, 55440-1022, US

(73) Assignee: Transoma Medical, Inc.

(21) Appl. No.: 12/165,361

(22) Filed: Jun. 30, 2008

(51) Int. Cl. A61N 1/00

(52) U.S. Cl. 607/3

(57) ABSTRACT

In a method of providing therapy to a patient to prevent an occurrence of a dangerous cardiac event, a cardiac signal sensed over multiple cardiac cycles is received. A risk of impending cardiovascular insult is determined, using the received cardiac signal, by assessing an indicator of proarrhythmogenic substrate and a change in sympathovagal balance. A therapy comprising acupuncture to modulate sympathovagal balance is administered based on the determined risk.

### TECHNICAL FIELD

This disclosure relates to predicting an upcoming cardiovascular insult, and taking measures to prevent its occurrence.

### BACKGROUND

One such prophylactic approach involves patient implantation with an implantable cardiac defibrillator (ICD). However, identifying candidate patients in the prophylactic population likely to benefit from an ICD has proven challenging. For example, conventional risk stratification criteria for identifying patients for ICD implant has resulted in only one life saved for every 14 to 17 ICDs implanted. Also, limitations of present fibrillation detection algorithms can result in false positive detections, so that today about 10-20% of delivered defibrillation shocks are inappropriate. Moreover, about 50% of SCD cases occur in patients with compromised cardiac function, though not to the level typically required for ICD indication. These patients—having compromised cardiac function and at-risk for SCD, yet not indicated for ICD implantation—may benefit from a system that identifies risk of impending cardiac insult, such as arrhythmias that may lead to SCD, and provides a preventative therapy so that action may be taken to thwart its occurrence.

It is known that patients with heart disease exhibit increased sympathetic drive to neurohormonal receptors in the heart and elsewhere. This increased sympathetic drive exerts an adverse effect on the cardiovascular system, and can lead to lethal arrhythmias (i.e., SCD) and exacerbate ischemia and pump failure. Studies suggest sympathovagal imbalance may trigger fatal arrhythmias during acute myocardial ischemia, thus resulting in sudden death., see e.g., Andrea Pozzati et al., Transient Sympathovagal Imbalance Triggers “Ischemic” Sudden Death in Patients Undergoing Electrocardiographic Holter Monitoring, J. Am. C. Cardiology (Mar. 15, 1996) 27:847-52. In some patients, autonomic imbalance may trigger electrical storms, which may be defined as multiple life-threatening arrhythmias in a twenty-four-hour period.

Within a clinical setting, acute sympathetic blockade has been successfully applied to treat electrical storms. See Koonlawee Nademanee et al., Treating Electrical Storm. Sympathetic Blockade Versus Advanced Cardiac Life Support-Guided Therapy, Circulation (Aug. 15, 2000) 102:742-47. Also, electroacupuncture and magnetoacupuncture have been shown to have beneficial effect by decreasing myocardial ischemia and reducing ventricular arrhythmias associated with ischemia by reducing sympathetic outflow. See John Longhurst, Electroacupuncture Treatment of Arrhythmias in Myocardial Ischemia, Am. J. Physiological Heart Circulatory Physiology, (Jan. 19, 2007) p S0363-6135. These treatment examples have been used in clinical settings upon onset or progression of cardiovascular insult.

### SUMMARY

A cardiac or cardiovascular signal may be measured and analyzed to determine a risk of impending cardiovascular insult. Based on the determination, a therapy that modulates sympathovagal balance may be administered to thwart the occurrence of the cardiovascular insult.

In a first general aspect, a method of providing therapy to a patient to prevent an occurrence of a dangerous cardiac event includes receiving a cardiac signal sensed over multiple cardiac cycles. The method also includes determining, using the received cardiac signal, a risk of impending cardiovascular insult by assessing an indicator of proarrhythmogenic substrate and a change in sympathovagal balance. The method further includes administering, based on the determined risk, a therapy comprising acupuncture to modulate sympathovagal balance.

In various implementations, the method may further include detecting, using the received cardiac signal, a cardiac instability trigger for use in determining the risk of cardiovascular insult. The cardiac instability trigger may be one or more ectopic beats. The indicator of proarrhythmogenic substrate may be an altemans burden that exceeds a predetermined threshold, a repolarization altemans burden determined from the cardiac signal, a QRS altemans burden determined from the cardiac signal, or a mechanical alternans burden determined from the cardiac signal. The indicator of proarrhythmogenic substrate may be a hypertension burden determined from the cardiac signal, one or more delayed afterdepolarizations, determined from the cardiac signal, that exceed a predetermined threshold, or a QT interval change or ST segment deviation that exceeds a predetermined threshold. The change in sympathovagal balance may be estimated from a heart rate variability measurement determined from the cardiac signal, or from a heart rate increase measurement determined from the cardiac signal. The therapy can modulate sympathovagal balance by activating parasympathetic drive. The acupuncture may comprise electro-acupuncture applied to one or more cardiovascular acupoints, or magneto-acupuncture applied to one or more cardiovascular acupoints. The cardiac signal may be an electrocardiogram signal or a blood pressure signal. The impending cardiovascular insult may be arrhythmia or acute myocardial ischemia. The method may further include initiating an alarm to alert an emergency responder to the impending cardiovascular insult, or issuing an alert in a manner that allows a preventative action to be taken to prevent the dangerous cardiac event. The preventative action may include donning a wearable external defibrillator, and the patient may perform the action in response to the alert.

In a second general aspect, a system for providing therapy to prevent an occurrence of a dangerous cardiac event includes a monitoring component that receives a cardiac signal sensed over multiple cardiac cycles. The system also includes a processing component that determines, using the received cardiac signal, a risk of impending cardiovascular insult by assessing an indicator of proarrhythmogenic substrate and a change in sympathovagal balance. The system further includes a therapy component that administers, based on the determined risk a therapy comprising acupuncture to modulate sympathovagal balance.

Some implementations may include one or more of the following advantages: an impending cardiac insult may be predicted and proactively treated to prevent its occurrence, patient longevity may be improved, patient quality-of-life may be improved, a low cost monitoring and therapy system may provide protection against adverse cardiac insults for patients not indicated for an ICD, a patient may be warned in advance of an impending cardiac insult so that preventative action may be taken.

The details of one or more implementations are set forth in the accompanying drawings and the description below. Other features, objects, and advantages will be apparent from the description and drawings, and from the claims.

### DESCRIPTION OF DRAWINGS

FIG. 1A is a conceptual diagram of an exemplary system that can be used to predict an impending cardiovascular insult and provide a therapy designed to prevent occurrence of the insult.

FIG. 1B is a conceptual diagram of another exemplary system that can be used to predict an impending cardiovascular insult and provide a therapy designed to prevent occurrence of the insult.

FIG. 1C is a conceptual diagram of an exemplary system that can be used to predict an impending cardiovascular insult, provide a therapy designed to prevent occurrence of the insult, and issue an alert.

FIG. 1D is a conceptual diagram of an exemplary system that can be used to predict an impending cardiovascular insult and provide a therapy designed to prevent occurrence of the insult, and initiate an alarm to alert an emergency responder.

FIG. 2 is a flow chart of an exemplary process that can be used to assess patient cardiac risk using cardiac signal data and provide a therapy or alert in advance of a predicted impending cardiovascular insult.

FIG. 3 is a flow chart of an exemplary process that can be used to calculate sensitivity of cardiac function to sympathetic drive.

FIG. 4 is another flow chart of an exemplary process that can be used to calculate sensitivity of cardiac function to sympathetic drive, and includes additional information as compared to the flow chart of FIG. 3.

FIGS. 5A, 5B, and 5C are exemplary charts of data trended over time.

FIG. 6 is a series of exemplary charts that can be used to assess a patient's cardiac risk profile.

FIG. 7 is a block diagram of an exemplary device that can be used to predict an upcoming cardiovascular insult, and taking measures to prevent its occurrence.

FIG. 8 is a simplified block diagram of an exemplary implantable device.

Like reference symbols in the various drawings indicate like elements.

### DETAILED DESCRIPTION

Described herein are methods, systems, and devices that can be used to assess or determine a risk of an impending cardiac insult or dangerous cardiac event for a patient, and provide a therapy to prevent the occurrence of the impending cardiac insult. A cardiac or cardiovascular signal, such as a physiologic signal that is influenced by the patient's cardiac cycle or by cardiac parameters, can be measured or sensed. In an implementation, it can be determined that the patient may be likely to suffer a serious cardiac insult within a short period of time, based on determinations made using the cardiac signal. For example, it may be determined that the patient is at a high risk of suffering a severe cardiac or cardiovascular insult, such as an arrhythmia or acute myocardial ischemia that may result in sudden cardiac death, within about ten, fifteen, twenty, twenty-five, or thirty minutes from the time of the cardiac signal measurement. Based on this determined risk, a therapy can be administered that modulates sympathovagal balance within the patient to prevent occurrence of the dangerous cardiac event, according to an implementation. Because the techniques can be used to predict an impending cardiovascular insult, arrhythmias, ischemia, or other acute cardiac injuries that can lead to sudden cardiac death may be averted.

The therapy may include acupuncture (e.g., electro-acupuncture or magneto-acupuncture) applied to one or more acupoints located along body surface meridians or channels on the patient's body, and may modulate sympathovagal balance by activating parasympathetic drive, according to an implementation. The concept of “sympathovagal balance” is used to characterize the autonomic state resulting from sympathetic and vagal interactions. Activation of parasympathetic drive may decrease the patient's cardiac vulnerability and reduce the likelihood that the predicted cardiac event will occur. In this fashion, a risk of an impending severe cardiac event may be detected before the event occurs, and a timely therapy to prevent occurrence of the event may be proactively administered. Because the administered therapy may be far less painful than conventional ICD shock treatment in response to onset of a cardiac event, and because the life-threatening event may be averted by timely application of the therapy, patient quality-of-life and longevity may be improved, according to some implementations.

Without limitation, the cardiac or cardiovascular signal may be an electrical signal or a hemodynamic signal, and may be measured using implanted electrodes or sensors, or alternatively using external electrodes or sensors. Any appropriate number of electrodes or sensors may be used (e.g., one, two, three, four, etc.). In some implementations, a combination of internal and external electrodes, sensors, or some combination can be used to measure one or more such physiologic signals for use in evaluating a patient's risk of an impending cardiovascular insult.

Examples of such signals can include an electrocardiogram (ECG) signal, an electrogram (EGM) signal, a blood pressure signal, a blood flow signal, or a signal comprised of impedance measurements. The signal may be measured at various locations within or outside of the body, including within a heart chamber (e.g., left ventricle, right ventricle, left atrium, right atrium), at an implanted subcutaneous location outside of the heart (e.g., in a pectoral region), within a body vessel (e.g., within an artery or vein), within or across an organ, at an external location on the patient's skin, and others.

The cardiac signal can be analyzed to assess an indicator of proarrhythmogenic substrate and a change in sympathovagal balance, according to various implementations. Proarrhythmogenic substrate can be characterized by alteration in electrical properties of myocardial tissue that creates conditions for initiation and maintenance of arrhythmia. Often, it is pronounced in multiple conduction blocks that facilitate functional reentrant tachyarrhythmias that can degenerates into fibrillation in case of faster or multiple re-entry loops. The indicator of proarrhythmogenic substrate may be manifested as an alternans burden or a hypertension burden, for example, as a change in the QT interval of the cardiac cycle, or as one or more delayed afterdepolarizations, and may be associated with cardiac instability that renders the patient at risk of suffering a dangerous cardiac episode. The change in sympathovagal balance may be estimated from a heart rate variability measurement or a heart rate increase measurement, to list just a few examples, determined from the cardiac signal. The change in sympathovagal balance may also render the patient at increased risk of suffering a cardiovascular insult, such as if the change results from heightened sympathetic drive or withdrawn parasympathetic drive. As will be described more fully below, the combination of an indication of proarrhythmogenic substrate and a change in sympathovagal balance may provide a warning that a cardiac event capable of leading to sudden cardiac death may occur within a relatively short time, such as within an hour, a half-hour, twenty minutes, fifteen minutes, ten minutes, five minutes, or less.

In some implementations, a cardiac instability trigger may also be detected and used in the determination of risk of impending cardiac event. For example, a detected cardiac instability trigger, in combination with the indication of proarrhythmogenic substrate and a change in sympathovagal balance, may further increase the likelihood of an impending dangerous cardiac event in some implementations. Examples of such instability triggers can include one or more ectopic beats in the cardiac signal. Premature ventricular contractions, for example, may be a cardiac instability trigger.

The systems, devices, and methods described here may be used by a patient as the patient goes about her daily activities (i.e., in an ambulatory environment). As such, at the time that a determination of a high risk of an impending severe cardiac event is made, the patient may likely be away from a hospital or facility equipped to administer therapy or quickly provide medical attention should the event occur as predicted. By providing a therapy that modulates sympathovagal balance, an event such as an arrhythmia or acute myocardial ischemia that may result in sudden cardiac death for the patient may be averted. As a backup or in addition to the provided therapy, an alarm or warning message may be transmitted to alert an emergency responder of the patient's condition, according to some implementations. Alternatively or in addition, an alert may be issued to the patient, to a family member, a co-worker, or a bystander, etc., that allows a preventative action to be taken to prevent the dangerous cardiac episode, or to mitigate the effects of the episode if its occurrence cannot be prevented. Examples of such preventative actions may include donning, or assisting the patient with wearing or placing, a wearable external defibrillator or an acupuncture applicator so that preventative or reactive therapy can be administered. In some implementations, the patient may choose not to wear a therapy application device until such an alert is received, instead choosing to carry or have access to the therapy application device (e.g., as by carrying the device with them) and quickly placing the device in position for therapy administration upon receiving the alert.

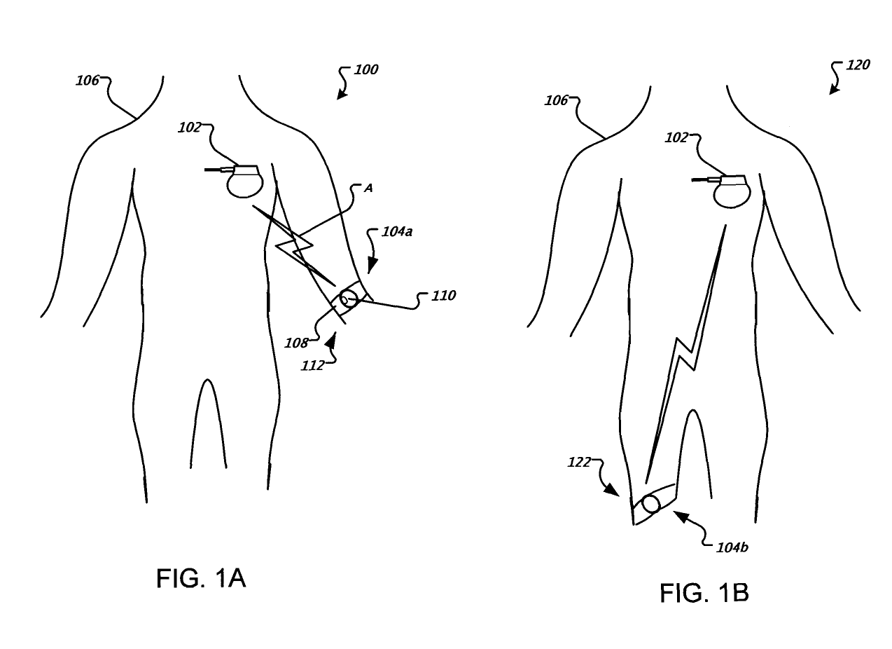

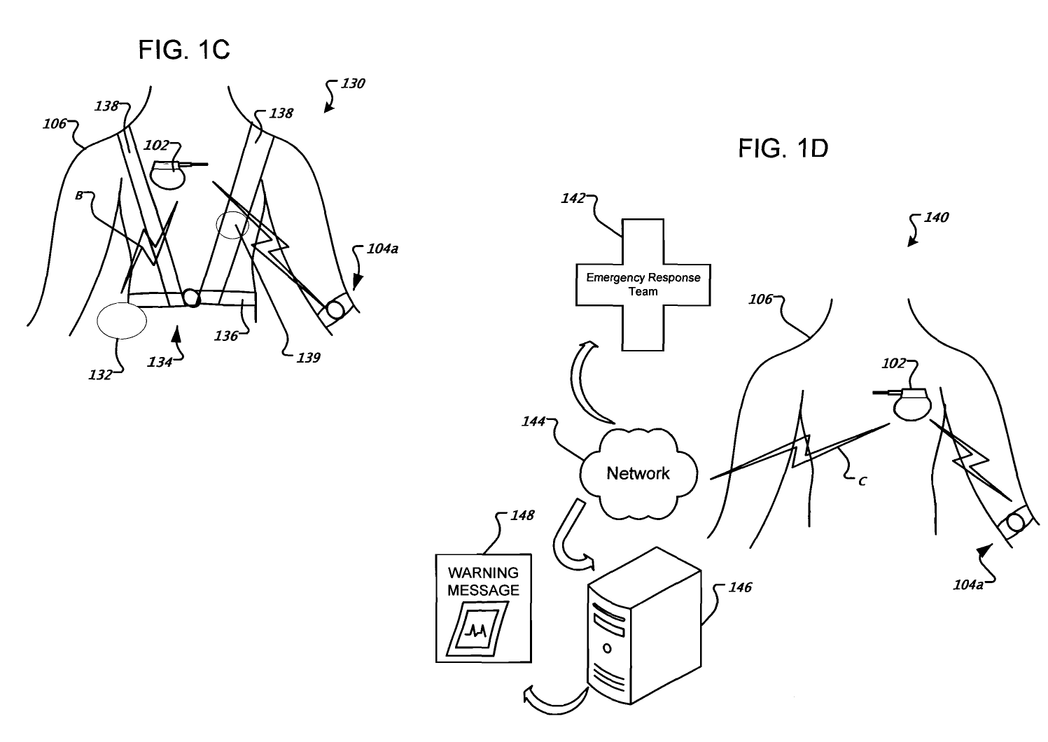

In some implementations, the methods described herein may be implemented entirely within one or more implantable devices. In other implementations, portions or all of the methods may be implemented in one or more external (i.e., non-implanted) devices, and in some cases a portion of a method may be implemented in one or more implantable devices and a remaining portion of the method may be implemented in one or more external devices. FIGS. 1A-1D, discussed below, depict exemplary implementations of systems that can be used to predict an impending cardiac insult and provide a therapy. Many variations are possible, including combining or separating aspects of the various implementations, or including additional or alternative features with any of the depicted implementations.

FIG. 1A is a conceptual diagram of an exemplary system 100 that can be used to predict an impending cardiovascular insult and provide a therapy designed to prevent occurrence of the insult. The system 100 includes an implantable device 102 and an external therapy device 104a. The implantable device 102 may be implanted within a patient 106, and may communicate wirelessly with the external therapy device 104a, as depicted by communication indicator “A” in FIG. 1A. In this illustrative implementation, the implantable device 102 is shown implanted subcutaneously in a left chest region of the patient 106. In some implementations, the implantable device 102 includes two or more electrodes for measuring physiologic electrical activity, such as an ECG or electrogram signal, or a body impedance. In some implementations, the implantable device 102 includes a sensor for measuring a hemodynamic signal, such as a pressure sensor for measuring a body pressure within the patient 106 or a blood flow sensor to measure blood flow through a body vessel. In some implementations, the implantable device 102 can measure both an electrical signal and a hemodynamic signal, each of which can be used in assessing risk of an impending severe cardiovascular insult.

In FIG. 1A, the patient 106 is shown with the implantable device 102 implanted in a subcutaneous pocket beneath the patient's skin. The device 102 includes a short lead, which may also be positioned subcutaneously, and which may include one or more sense electrodes and/or pressure or flow sensors. The device 102 also includes a housing that may include one or more sense electrodes on an exterior surface. In other implementations, the implantable device 102 can be implanted such that one or more connected leads extend into the patient's heart. In still other implementations, the device 102 is an external device, not implanted within the patient 106, such as an external ECG-sense device with electrodes for attachment to the patient's skin, or an external device for measuring an internal patient body pressure (e.g., blood pressure).

The external therapy device 104a is depicted as a wrist-worn device, and may include a band or attachment member 108 (e.g., a strap) for securing the device 104a at a desired external body location, and an acupuncture delivery component 110 coupled to the attachment member 108. In various implementations, the acupuncture delivery component 110 may be configured to administer electro-acupuncture or magneto-acupuncture to the patient 106, and may include stimulation circuitry for providing electrical stimulation or magnetic field stimulation as appropriate. One, two, three, four, or more electrodes may be disposed on an external surface of the acupuncture delivery component 110, such that when the therapy device 104a is positioned on the patient the one or more electrodes may deliver the acupuncture therapy to the body 106. The one or more therapy electrodes may be in contact with the patient's skin, and the therapy device 104a may be worn or positioned such that stimulation from the delivery component 110 acts on a cardiovascular acupoint. The delivered therapy to the cardiovascular acupoint may activate parasympathetic drive within the patient 106, which may modulate sympathovagal balance and reduce the patient's risk of an adverse cardiac event.

As described above, the acupuncture delivery component 110 may be positioned to deliver therapy at or near an acupuncture point or acupoint of the patient, and many variations are possible. In FIG. 1A, the external therapy device 104a is shown about the wrist 112 of the patient, so that the acupuncture delivery component 110 may deliver therapy in the vicinity of the P6 acupoint, which is associated with a median nerve that generally runs along the inside center of the patient's forearm, just beneath tendons of palmoris longus and flexor carpi radialis. The P6 acupoint is located one-sixth of the distance from the distal wrist crease to the cubital crease, between the tendons mentioned above. The acupuncture deliver component 110 may be positioned on the ventral side of the wrist above the P6 acupoint so that the one or more stimulation electrodes are in contact with the skin for delivery of stimulation to the acupoint.

FIG. 1B is a conceptual diagram of another exemplary system 120 that can be used to predict an impending cardiovascular insult and provide a therapy designed to prevent occurrence of the insult. The system 120 is similar to the system 100 of FIG. 1A, except that the external therapy device 104b is located near a knee 122 of the patient 106. Device 104b may be similar or identical to device 104a described above, and may stimulate acupuncture point GB34, about one inch below the patient's knee. The GB34 point is along the gall bladder meridian and, like the P6 acupoint described above with reference to FIG. 1A, is known to respond to acupuncture therapy with positive cardiac therapeutic effect.

It is contemplated that an external therapy device 104 with an acupuncture delivery component 110 may be used to deliver acupuncture therapy at any of the known acupoints, which generally lie along various meridians. For example, acupoints associated with any of the twelve main meridians (e.g., associated with the bladder, gall bladder, heart, kidney, large intestine, liver, lung, pericardium, small intestine, spleen, stomach, and triple warmer) may be stimulated as described above with reference to the exemplary implementations shown in FIGS. 1A and 1B. The precise locations of acupoints associated with each of these meridians is known to a person having ordinary skill in the art, and for brevity will not be expanded upon here. In some cases, modifications to the external therapy device 104, such as alternative sizes, shapes, or configurations of the attachment member 108 or the acupuncture delivery component 110 may be used to better facilitate therapy delivery at the location of interest, as would be apparent to a person having ordinary skill in the art.

FIG. 1C is a conceptual diagram of an exemplary system 130 that can be used to predict an impending cardiovascular insult, provide a therapy designed to prevent occurrence of the insult, and issue an alert. The system 130 includes the implantable device 102 and the external therapy device 104a as described above with reference to FIG. 1A. In this illustrative implementation, the implantable device 102 is shown implanted in a right chest pectoral location. The system also includes an external communication device 132, which may communicate wirelessly with the implantable device 102 (indicated by communication indicator “B” in FIG. 1C), and may provide an alert or warning to the patient 106 of a severe cardiovascular insult predicted to occur in the near future. In some implementations, the communication device 132 may be a handheld device such as a mobile phone, personal digital assistant (PDA), smartphone, pager, or the like that combines communication functionality with the implantable device 102 and ability to provide information to the patient with capability to perform one or more independent or unrelated functions (e.g., phone calls, internet access, various PDA functions, scheduling activities, e-mail receipt, delivery, and organization, event logging, etc.). The device 132 may be carried by the patient (e.g., in a pocket, bag or purse), attached to a belt, or otherwise kept in proximity to the patient so that the patient can be timely alerted to a predicted severe cardiac event by the implantable device 102. Various methods of notification are possible, including audible, visual, or tactile alerts. For example, the device may include a display screen that presents a message, a speaker over which an audible message or alarm is played, one or more lights that flash or otherwise visually alert the patient, or a vibrating element that agitates to alert the patient, to list just a few examples. Other notification methods are possible.

In response to receiving the alert, the patient 106 may take a preventative action designed to prevent occurrence of the dangerous cardiovascular episode. For example, the patient may don a wearable external defibrillator 134, illustratively shown in FIG. 1C as including a belt 136 and shoulder straps 138 to be worn like a vest. The external defibrillator 134 includes a defibrillation component 139 comprising defibrillation shock generation circuitry, delivery electrodes, and control circuitry for determining when to administer a defibrillation shock, as is well known in the art. Sense electrodes (not shown in FIG. 1C) may additionally be included for attachment to the patient's skin and monitoring heart rhythms. An example of an external defibrillator that may be used is the Zoll Lifecor LifeVest from the Zoll Lifecor Corporation of Pittsburgh, Pa. In the event that acupuncture therapy from the external therapy device 104a is not successful in thwarting the onset of a dangerous cardiac episode, the external defibrillator 134 may detect the event and deliver a defibrillation shock to the patient to depolarize the heart, terminate the fibrillation, and restore normal sinus rhythm.

FIG. 1D is a conceptual diagram of an exemplary system 140 that can be used to predict an impending cardiovascular insult and provide a therapy designed to prevent occurrence of the insult, and initiate an alarm to alert an emergency responder 142. The system 140 includes the implantable device 102 and the external therapy device 104a discussed above with reference to FIG. 1A. In this implementation, the implantable device 102 may wirelessly transmit information, illustrated by communication indicator “C” in FIG. 1D, over a network 144, which may include various networks and devices that comprise the Internet, for example, or alternatively may be a local area network (LAN) or a wide area network (WAN).

This information may include, for example, an alarm message for receipt by an emergency responder 142. This may provide backup coverage by enabling the emergency responder 142 to attend to the patient 106, if necessary. In various implementations, the emergency responder 142 may immediately attend to the patient 106, or may first attempt to contact the patient, as by phone, e-mail, text message, etc., to consult with the patient 106. In some cases, location information may be included with the information so that the patient may be expediently located. The location information may include GPS coordinates, for example, or an address or other location indicator. The location information may be provided by an external device in communication with the implantable device, for example. In some cases, the emergency responder 142 may receive separate transmissions from the implantable device (e.g., the warning message) and the external device (e.g., location information).

In some implementations, the implantable device 102 telemeters data to an intermediate external device (not shown in FIG. 1D), such as a handheld device (e.g., external device 132 in FIG. 1C) or a base station unit in the patient's home, and the intermediate external device sends the data over the network 144. The system 140 optionally includes an external computing device 146, shown in FIG. 1D as a server device, which may implement portions or all of the cardiac risk assessment methods described herein, and which may communicate with the implantable device 102 over network 144, possibly through an intermediate external device as discussed above. The external computing device 146 may be located at a hospital or a care service center, for example.

In some implementations, the implantable device can telemeter the measured cardiac signal or information derived from the measured signal for analysis outside of the implantable device 102. Such analysis might occur in the external device 132 (FIG. 1C), in the external therapy device 104, or in the external computing device 146. In some cases, portions of the methods described herein are implemented in one device, and portions are implemented in one or more separate devices, such as any of the devices mentioned above.

After sensing the cardiac signal data, the implantable device 102 can store a sample of the cardiac signal data in internal memory. In some implementations, the device 102 may not record until a specified triggering event occurs. In some cases, the device 102 may evaluate the sensed signal data, and may determine whether or not to store the sensed data for later processing. For example, the device 102 may determine that the sensed data is corrupted by noise to such a degree that analysis results are likely to be unduly compromised, and may accordingly choose not to store the data in internal memory. In this fashion, memory space within the implantable device 102 may be conserved. Alternatively, the device may mark noisy data to indicate that processing of the data should be adjusted to account for the noise present with the stored data. Such noise can be caused by ectopic beats (for example, caused by premature ventricular contractions), uncorrelated beats, EMG signals, or electrical interference, to list just a few examples. However, in some cases such noise may be indicative or a cardiac instability trigger, as discussed above, such as when the noise is caused by ectopic beats. In these cases the information can be used in the risk assessment. In some implementations, the sensing device 102 can sense or measure the cardiac signal data and transmit the cardiac signal data without internally storing the data.

Data (e.g., cardiac data), alarms, messages, etc., may be presented to a health care professional or emergency responder, as by displaying the data or information on a display screen of a monitoring device 148 at a hospital, care center, remote monitoring facility, emergency response center, mobile location, or the like. The information may be presented in any number of ways. The monitoring device 148 may receive information from the external computing device 146 over a wired or wireless communication connection, including over a local area network, a wide area network, or the Internet.

The monitoring device 148 can include a program that displays the data or information graphically on a display device. Graphical or textual information may be presented, as well as audible or tactile information, or combinations of the foregoing, depending upon the implementation. For example, the monitoring device 148 may display a graph of cardiac information, and the health care professional may interpret the data to make an assessment. Alternatively, the monitoring device 148 can display the data using numeric or text-based means. The data can include a warning message, location information, a severity indicator comprising a likelihood of a severe cardiac event, timing information, such as a predicted time or interval in which the event is likely to occur, and the like. In various implementations, any of the above information (including warnings) can be presented to the patient, such as via external communication device 132 (see FIG. 1C).

In some implementations, the external computing device 146 can send an e-mail or other communication (phone call, text message, SMS message, pager signal, etc.) to the health care professional or emergency responder when an issue arises, such as if a high risk of impending cardiac event is predicted based on the cardiac signal data. In these cases, immediate medical attention may be summoned, or pre-emptive therapy measures may be initiated. Similarly, such a message may be alternatively or simultaneously communicated to the patient 106 (via e-mail, phone call, text message, SMS message, pager signal, etc.), to encourage the patient to seek medical attention or initiate medication or therapeutic measures. The computing device 146 is shown as a computer (e.g., a server, a desktop, laptop, or client-type computing device) in FIG. 1D, but in some implementations the device 146 can be a hand-held or mobile device able to receive wireless communications, such as a mobile phone, smartphone, or PDA. The device 146 can also be a device worn or carried by the patient 106 (or physician, e.g.). The communications between any of the devices described above with reference to FIGS. 1A-1D may be bidirectional or unidirectional, according to various implementations.

In the implementations shown in FIGS. 1A-1D, the implantable device 102 is depicted as a monitoring device, but in some implementations the device may also include therapeutic functionality that can be used in addition to, or in lieu of, the therapy provided by the external therapy device 104. Alternatively, the implantable device 102 may be communicably connected to a separate implantable therapy device (not shown in FIGS. 1A-1D), which may be implanted within the body 106 at an appropriate location. Without limitation, such therapeutic functions or devices may include drug delivery or a drug pump, an implantable cardio-defibrillator device, a pacing device, including a device enabled for anti-tachycardia pacing, a neurostimulator, a topical medication applicator, and others. In various implementations, the cardiac assessment of the patient's risk profile can be used to automatically initiate or modify an administered therapy to the patient, such as by one of the devices mentioned above.

In some examples, an administered medication may modulate sympathovagal balance by sympathetic blockade. Examples of medications that may induce sympathetic blockade include esmolol and propanolol. A drug pump for dispensing these or other medications may be implantable or external to the patient, and may be in communication with the implantable device 102 over a wired or wireless connection.

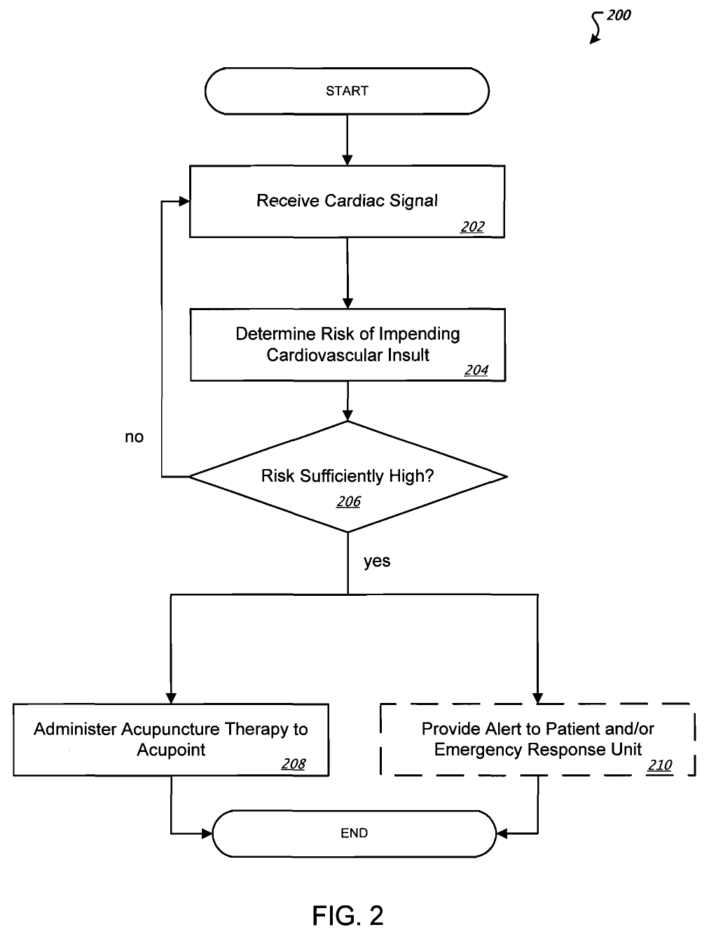

FIG. 2 is a flow chart of an exemplary process 200 that can be used to assess patient cardiac risk using cardiac signal data and provide a therapy or alert in advance of a predicted impending cardiovascular insult. At step 202, a cardiac signal is received. The signal can be measured or received following various triggers. The cardiac signal may be a physiologic signal that is associated with the patient's cardiac cycle. The cardiac signal may be an electrical signal or a hemodynamic signal. Examples of electrical cardiac signals that can be measured include an ECG signal, an electrogram signal, or portions of such signals (e.g., the QRS complex, the repolarization wave, or subsets thereof). Such electrical cardiac signals may be measured by two or more sense electrodes within or outside the body of the patient. Examples of hemodynamic signals that can be measured include pressure signals, such as a blood pressure signal within a chamber of the heart or within the patient's cardiovascular system (e.g., within an artery or a vein), or taken using an external sense measurement (e.g., traditional arm pressure cuff), or a blood flow signal, such as a blood flow measurement taken within or across an artery or vein, for example. As another example, an electrical impedance may be measured, as by injecting a known current, measuring the resulting induced voltage (or alternatively, providing a known voltage and measuring current), and computing associated impedance according to Ohm's law.

Additional examples of implantable devices capable of measuring an internal body pressure are provided in U.S. patent application Ser. No. 10/077,566, filed Feb. 15, 2002, and titled “Devices, Systems and Methods for Endocardial Pressure Measurement,” and U.S. Patent No. 6,033,366, titled “Pressure Measurement Device,” and U.S. Pat. No. 6,296,615, titled “Catheter With Physiological Sensor,” the entire disclosures of which are herein incorporated by reference in their entirety. Additional examples of implantable devices capable of measuring an impedance are provided in U.S. patent application Ser. No. 11/933,872, filed Nov. 1, 2007, and titled “Calculating Respiration Parameters Using Impedance Plethysmography,” the entire disclosure of which is herein incorporated by reference in its entirety. Additional examples of implantable devices capable of measuring electrical cardiac signals, such as ECG signals, are provided in U.S. patent application Ser. No. 11/119,358, filed Apr. 28, 2005, and titled “Implantable Medical Devices and Related Methods,” the entire disclosure of which is herein incorporated by reference in its entirety. In some implementations, the signal may be sensed or measured and transmitted by a first device for receipt by a second device, such as over a wired or wireless communication channel.

Some implementations can bin (i.e., store the information according to a particular feature) cardiac data according to various parameters. For example, the cardiac data can be binned according to time of day that the data is collected. In some implementations, measured cardiac data can be binned by heart rate associated with the data sample. In other implementations, the cardiac data can be binned according to the components of cardiac data collected, such as T-wave alternans information (or QRS or mechanical alternans information, etc.), heart rate variability information, etc., as appropriate. In still other implementations, the cardiac data can be binned according to a type of therapy the patient is undergoing while the ECG data is collected in cases where an ongoing therapy regimen is being followed.

Next, at step 204, a risk of an impending cardiac insult is determined. In some implementations, the risk may be determined by assessing the cardiac signal for an indication of a proarrhythmogenic substrate and a change in sympathovagal balance. This combination of factors may be indicative of a likelihood of a severe cardiac episode within the near future, such as within about 10-20 minutes. The measured cardiac signal can include information from multiple cardiac cycles at multiple periods in time, and the risk assessment can be made using information from several or all of cycles or periods. The indicator of proarrhythmogenic substrate may be manifested within the cardiac signal as an alternans burden over a period of time, as a hypertension burden over a period of time, as a QT interval change, or as one or more delayed afterdepolarizations that exceed a predetermined threshold.

Several types of alternans burdens can be determined. The alternans burden can be determined by analyzing the cardiac signal, or a portion of the cardiac signal, for periodic variability in the signal that may be indicative of cardiac instability. The periodic variability may be referred to as “alternans” of the physiologic signal. For example, an alternans amplitude in a 2:1 pattern (i.e., ABABAB . . . ) can be measured as a composite of amplitudes at frequencies that are odd multiples of one half of the heart rate (HR) (e.g., at 0.5*HR, 1.5*HR, 2.5*HR, etc.).

In some implementations, the physiologic or cardiac signal can be an ECG signal, or a portion of an ECG signal. For example, the method can be used to analyze an ECG signal, and specifically the T-wave or repolarization wave of the ECG signal, to determine if a patient exhibits T-wave alternans, a periodic variability associated with the T-wave of the ECG signal. In another example, the method can be used to analyze another portion of the ECG signal, such as the QRS complex, to detect QRS alternans associated with the QRS complex. In other implementations, an alternans burden associated with yet another portion of the ECG signal can be computed. As will be described further below, determination of the alternans burden may be used in assessing the patient's risk profile or to predict a patient's risk of sudden cardiac death. In other implementations, the method can be used to analyze a hemodynamic signal. Examples of methods that can be used to analyze alternans of a physiologic signal can be found in U.S. Provisional Application No. 60/991,650, filed Nov. 30, 2007, and titled “Physiologic Signal Processing To Determine A Cardiac Condition,” the contents of which are herein incorporated by reference in its entirety.

In implementations where the measured cardiac signal is a hemodynamic signal, the alternans burden may be a mechanical alternans burden determined from the hemodynamic signal. For example, in some implementations the measured cardiac signal is a blood pressure signal or a blood flow signal, either of which may be analyzed to determine a mechanical alternans burden. The mechanical alternans burden may be indicated by alternans (e.g., mechanical pulsus alternans) present in the hemodynamic signal, for example at frequencies that are odd multiples of one half of the heart rate.

In implementations where a hypertension burden is determined, the hypertension burden may be due to elevated systolic blood pressure, elevated diastolic blood pressure, or elevated systolic pressure and elevated diastolic pressure. In various implementations, pressure may be measured within a heart of the patient or outside the heart of the patient, such as within the vasculature of the patient (e.g., within an artery or vein), or even using an external pressure measurement apparatus. Also, impedance can be a surrogate for blood pressure or flow in various implementations, and a mechanical alternans burden may be determined from a signal comprised of multiple impedance measurements.

QT interval changes that exceed a predetermined threshold and delayed afterdepolarizations that exceed a predetermined threshold can also be indicators of a proarrhythmogenic substrate. The QT interval is the interval between the Q-wave and the T-wave in an ECG or EGM signal. A duration of the QT interval reflects a duration of ventricular recovery time. Both long and short QT and QT interval dispersion are associated with increased risk for arrhythmia. QT interval abnormalities may be congenital or acquired, resulting from electrolyte imbalance (especially hypokalaemia and/or hypomagnesaemia), endocrine dysfunction (e.g. hypothyroidism), autonomic imbalance, various disease states or most frequently, following clinical administration of drugs. Because the QT interval is modulated by the autonomic nervous system, changes with heart rate might affect its duration. As such, the measurement can be corrected for heart rate in some implementations.

Delayed afterdepolarization or late potentials are low amplitude, high-frequency electrical signals at the end of the QRS complex. Late potentials correlate with local areas of delayed activation in a working ventricular myocardium. Such local areas may constitute part of the arrhythmogenic substrate required to initiate and sustain reentry. Presence of late potentials was identified as an independent predictor of SCD in survivors of acute myocardial infarction.

In some implementations, an indication of a proarrhythmogenic substrate can be determined from a combination of the above indications. In the example of a measured ECG signal, for example, a repolarization alternans burden and a QRS alternans burden may be determined, and combined to form an alternans burden representative of both the repolarization burden and the QRS burden. Further, QT interval changes and/or late afterdepolarizations may be considered with either or both of the above burdens for a more global assessment. In similar fashion, in cases where the measured cardiac signal is a hemodynamic signal (e.g., a blood pressure signal), a determined mechanical alternans burden may be combined with a determined hypertension burden to form a hemodynamic burden representative of both the mechanical alternans burden and the hypertension burden.

In some implementations, two or more cardiac signals (e.g., an ECG signal, blood pressure signal, blood flow signal, or signal comprised of impedance measurements) can be measured, and one or more cardiac function determinations of a proarrhythmogenic substrate can be made from each of the two or more cardiac signals, using any two or more of the indicators described above.

In various implementations, sympathetic drive can be estimated from a heart rate variability measurement or parameter determined from the cardiac signal, and changes in sympathovagal balance can be tracked and monitored. In some implementations, sympathetic drive can be estimated from a heart rate increase measurement determined from the cardiac signal. Heart rate can be tracked and trended over time, and changes in a patient's heart rate can be used to refine calculations disclosed herein.

In some implementations, analysis may focus on periods where heart rate is increasing or accelerating, as such periods may be especially relevant for predicting patient cardiac instability. For example, a study by Narayan and Smith found that T-wave alternans (TWA) observed during periods of heart rate acceleration were more accurate in predicting ventricular tachycardia inducibility, see 35 J. Am. C. Cardiology, 1485, 1485-92. The study also showed that elevated TWA during a heart rate deceleration phase has lower predictive value, see id. In an implementation, heart rate history (e.g., acceleration and deceleration) can be tracked, and data corresponding to periods where heart rates are accelerating or decelerating can be analyzed separately, possibility using different analysis methods. For example, an alternans burden computation may be adjusted depending upon whether heart rate is accelerating or decelerating. In some cases, analysis may be adjusted during periods where heart rate is decreasing or decelerating, such as when the heart rate decrease drops below a threshold value after having been above the threshold for a predetermined time. This adjustment may account for a hysteresis effect in repolarization alternans, for example, that may occur during periods of recovery from a high-heart-rate state.

Based on the assessment of proarrhythmogenic substrate and the assessment of sympathovagal balance, a risk value may be assigned corresponding to a probability of a severe cardiac event occurring within a predetermined short-term time period, such as within about ten, fifteen, twenty, twenty-five, or thirty minutes. If the risk value does not exceed a predetermined threshold at step 206, the process returns to step 202 and resumes monitoring the cardiac signal.

If, however, the risk value exceeds a predetermined threshold at step 206, an acupuncture therapy may be administered at step 208 to an acupoint on the patient's body. The acupuncture therapy may modulate sympathovagal balance within the patient by activating parasympathetic drive, according to some implementations, which may reduce the likelihood of a severe cardiac episode occurring. Electroacupuncture, magnetoacupuncture, or traditional, needle-based acupuncture may be used. With electroacupuncture, a mild electrical current, for example about 2-4 mA, can be applied via electrodes in contact with the patient's skin to stimulate the acupoint.

Optionally, an alert can be provided to an emergency response unit and/or the patient at step 210. An alert to an emergency responder may indicate the patient's heightened risk of a severe cardiac event in the near future. Additional information, such as location information, patient medical history, medication information, and the like may also be included in some implementations. In some implementations, a tiered alert or alarm protocol may be implemented. For example, upon assessment of a high risk of near-term cardiac insult at step 206, therapy may be administered at step 208 and monitoring of the cardiac signal may continue at step 202. After a predetermined time, a new risk determination may be made at step 204. If the patient continues to indicate a sufficiently high risk of an adverse cardiac event, an emergency response team may immediately be summoned at step 210. Alternatively, if the patient indicates a sufficiently reduced risk of the adverse cardiac event, for example if the administered therapy is having beneficial cardiac effect, a physician or care provider message may be sent instead of summoning an emergency responder. The message may provide details of the cardiac signal data or the risk assessment, as well as therapy details and follow-up cardiac signal measurement information according to some embodiments. In this fashion, monitoring and risk assessment may continue following initial or ongoing administration of therapy, such that the patient's condition can be monitored for improvements or other changes, and an appropriate response can be taken.

An alert to the patient may instruct the patient to take a preventative action in anticipation of a severe cardiac event occurring within a short period of time. For example, the alert may instruct the patient to (or the patient may be trained to) wear an external defibrillator device (such as a wearable AED). As another example, the patient may apply or wear an acupuncture delivery device so that acupuncture therapy can be administered, which may be manually initiated or automatically initiated according to various implementations. In some cases, an implantable device may command the newly placed acupuncture device to administer therapy, such as by a wireless RF communication. In other cases, another person may implement some or all of the manual preventative steps, such as if the patient has become incapacitated or otherwise unable to comply with instructions associated with the alert.

As described above, several candidates are available as indicators of a proarrhythmogenic substrate. One such candidate is repolarization or T-wave alternans, which can indicate repolarization instability. Repolarization instability may be indicative of a proarrhythmogenic substrate. One study identified a substantial increase in T-wave alternans magnitude shortly before onset of a ventricular tachyarrhythmia, see V. Shusterman et al., Upsurge in T-Wave Alternans and Nonalternating Repolarization Instability Precedes Spontaneous Initiation of Ventricular Tachyarrhythmias in Humans, Circulation, (2006), 113: 2880-87. The study showed that T-wave alternans increased before the onset of an initiation of a ventricular tachyarrhythmia, and reached a peak value ten minutes before the event. See id. The increase was highly pronounced relative to the readings one hour and two hours preceding the event. See id.

When determining a risk value for the patient, an indicator of proarrhythmogenic substrate and a change in sympathovagal balance, such as an increase in sympathetic activity, may be considered. Each can be determined from the measured cardiac signal, according to various implementations. Proarrhythmogenic substrate determinations can be made considering an alternans burden (e.g., T-wave or repolarization alternans, QRS alternans, or a mechanical alternans burden), a hypertension burden, one or more delayed afterdepolarizations exceeding a threshold, or QT interval changes exceeding a threshold. Sympathovagal balance may be estimated from a heart rate variability measurement or from a heart rate increase measurement. Optionally, a presence of a cardiac instability trigger, such as one or more ectopic beats (e.g., caused by premature ventricular contractions) may also be used in the risk determination. The algorithm considers that the combination of a proarrhythmogenic substrate and a change in sympathovagal balance favoring sympathetic activity may leave the patient vulnerable to cardiac insult, especially if a cardiac instability trigger is also present. In these cases, a risk of a ventricular arrhythmia or acute myocardial ischemia which could result in sudden cardiac death may be heightened.

Upon administration of the acupuncture therapy, the patient's sympathovagal balance may be modulated, for example by parasympathetic activation, back to a level that is benign so that the predicted cardiac event may be averted, according to some implementations. Patients who may benefit from the systems and methods disclosed herein include those at risk of sudden cardiac death, especially those who are not currently implanted with or indicated for an ICD. Also, even patients implanted with an ICD but who experience frequent electrical storms, for example post-ICD shock, may benefit from the systems and methods disclosed here.

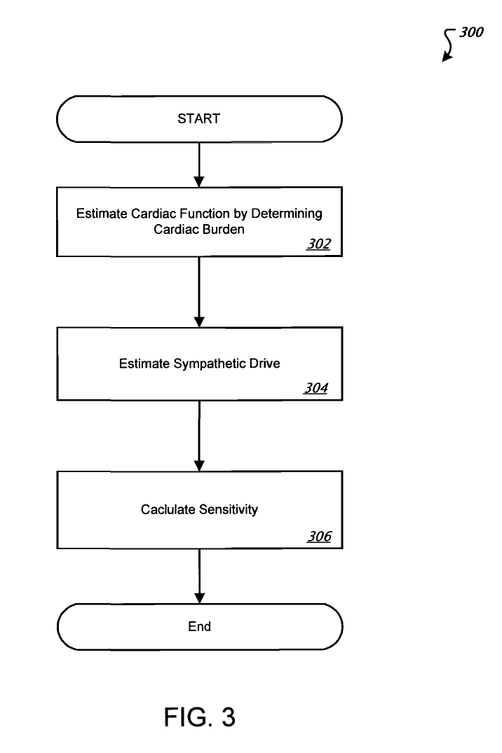

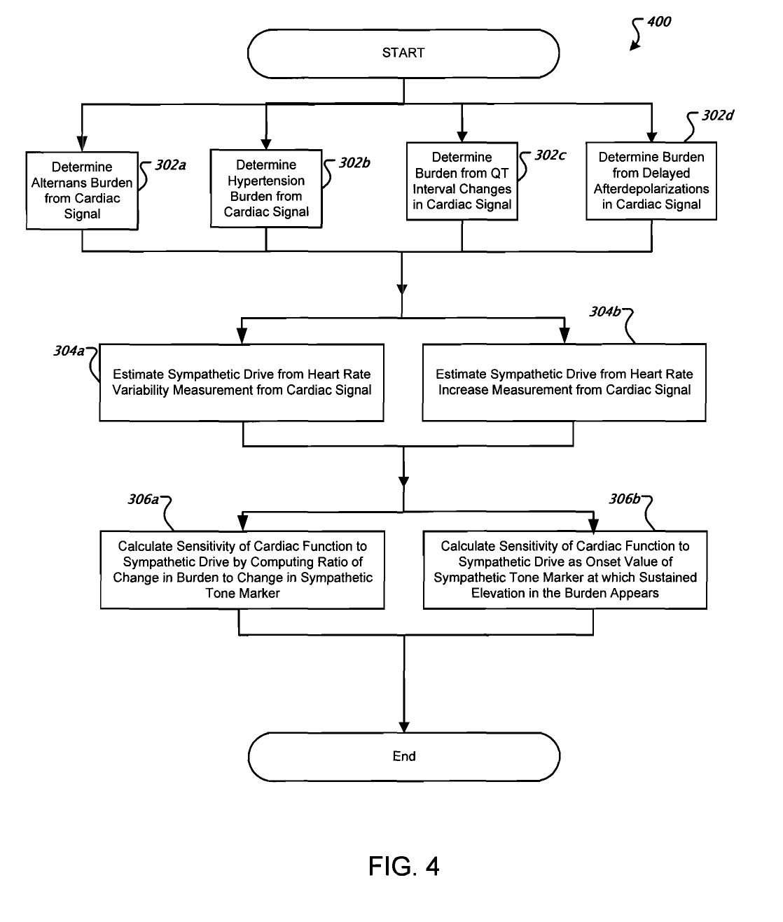

FIGS. 3 and 4 are flow charts of exemplary processes 300 (FIG. 3), 400 (FIG. 4) that can be used to calculate sensitivity of cardiac function to sympathetic drive. In general, the process 400 of FIG. 4 provides additional detail relating to the process 300 of FIG. 3, and includes various options for performing the steps of the FIG. 3 process 300, as will be described in further detail below.

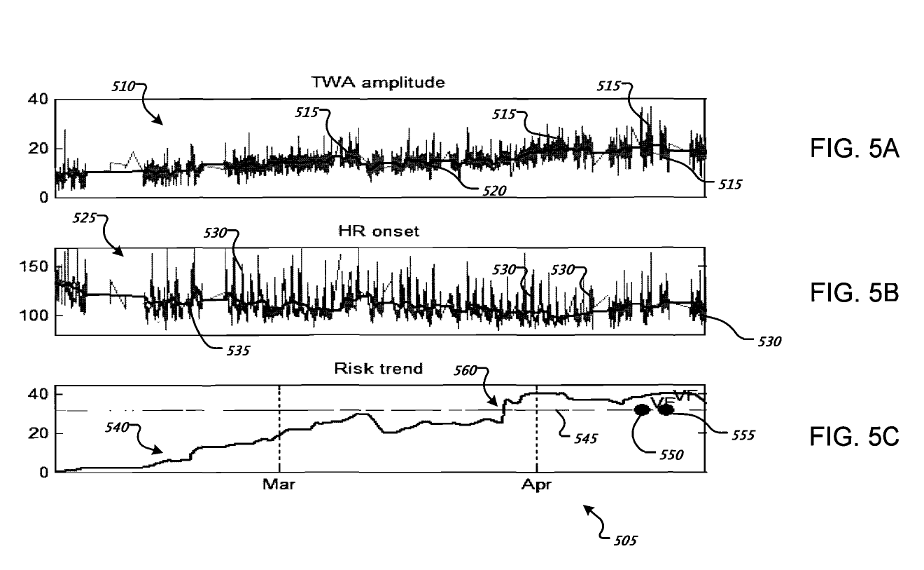

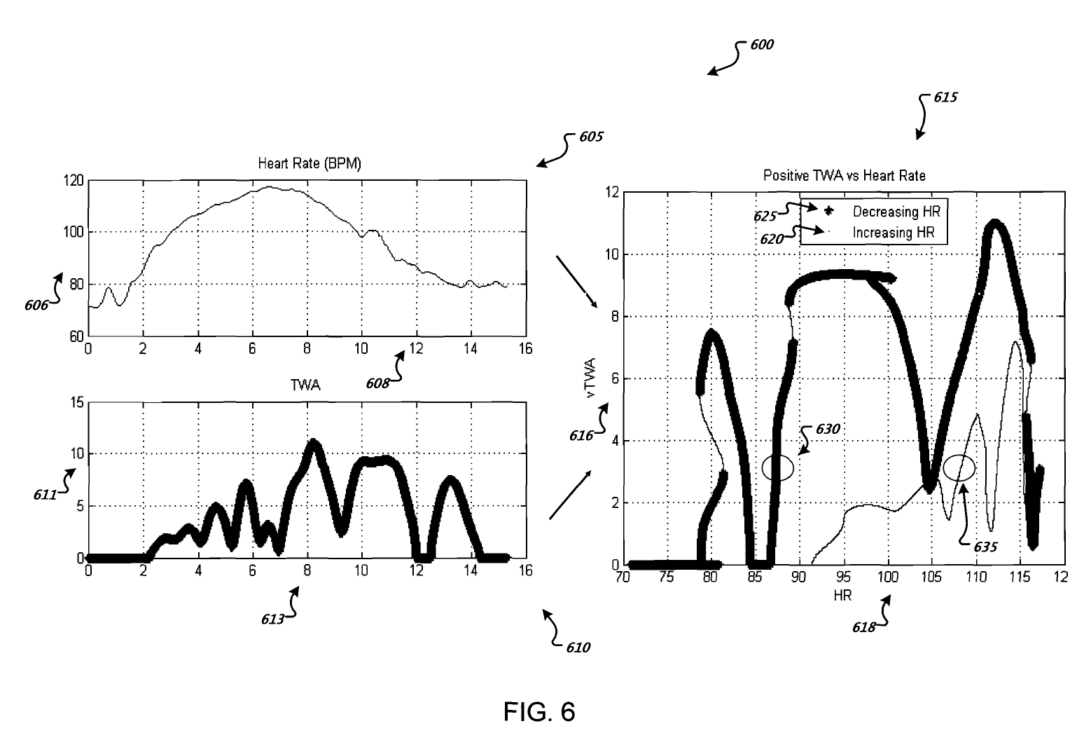

Referring first to FIG. 3, a cardiac function is estimated by determining a cardiac burden at step 302. The cardiac burden may represent an indicator of cardiac instability or patient vulnerability. In some cases, such instability may be caused by ischemia or other cardiac myopathies. Processing is performed on the measured cardiac signal, whether an electrical or hemodynamic signal. The cardiac burden may be determined over a period of time by considering strips of cardiac signal data measured periodically over the given time period. For example, the burden may be determined over several minutes when monitoring for an acute cardiovascular insult, or over several hours, days, weeks, or years when monitoring for cardiac conditions using longer-term trends. Examples will be described below with reference to FIGS. 5-6. In various implementations, cardiac signal data may be measured intermittently, such as for a predetermined time (strip length) at predetermined interval periods. Also, in some implementations cardiac signal data may be measured in response to a detected physiologic trigger, or in response to a manual trigger, as might be initiated by the patient or by a health care provider.

FIG. 4 shows that the cardiac burden may be determined from an alternans burden determined from the cardiac signal (302a), from a hypertension burden determined from the cardiac signal (302b), from QT interval changes determined from the cardiac signal (302c), or from delayed afterdepolarizations determined from the cardiac signal (302d). For example, a repolarization alternans burden may be determined from an ECG (or electrogram) signal or from a repolarization signal comprised of extracted T-waves from the ECG signal. Without limitation, such a signal may take any number of forms, such as a continuous signal formed from the extracted T-waves, or a discrete signal, such as a matrix of successive T-waves. Similarly, a QRS alternans burden may be determined from the ECG signal or from a QRS signal extracted from the ECG signal (e.g., continuous or discrete). In implementations where the measured cardiac signal is a hemodynamic signal (e.g., blood pressure, blood flow, or impedance), a mechanical alternans burden may be determined by detecting alternans in the signal, such as alternating high and low systolic blood pressure.

A hypertension burden may be determined from the hemodynamic cardiac signal. The hypertension burden may be due to elevated diastolic blood pressure, elevated systolic blood pressure, or a combination of the two. In various implementations, a measured impedance signal may serve as a surrogate for a blood pressure or flow signal. In this fashion, the impedance signal may be analyzed to determine an alternans burden as appropriate. In this case, cardiac risk may be evaluated by processing a surrogate for pressure without use of a pressure sensing transducer.

As described above, QT interval durations, ST segment changes or abnormalities, or delayed afterdepolarization can similarly be determined from the cardiac signal and used to determine the cardiac burden.

Next, sympathetic drive is estimated at step 304. Sympathetic drive may be estimated by considering the measured cardiac signal. As shown in FIG. 4, sympathetic drive may be estimated from a heart rate variability (HRV) measurement from the cardiac signal (304a), or from a heart rate increase measurement from the cardiac signal (304b). Sympathetic drive may be estimated so that a relationship between sympathetic drive and cardiac function may be trended and analyzed over time.

Heart rate, heart rate changes, or heart rate variability may be trended and tracked over time, and may be determined in a number of ways, according to various implementations. For example, within a given measurement strip of multiple cardiac cycles, heart rate can be determined by calculating a period between recurring periodic features of the cardiac signal, where the recurring periodic features correspond to a portion of the patient's cardiac cycle. For example, a period between successive R-waves of an ECG signal (i.e., the R-R interval) or between successive QRS complexes may be calculated. The heart rate frequency for particular cardiac data can be used to analyze multiple sets of cardiac data over time. In other implementations, heart rate variability or changes in heart rate can be determined as another autonomic parameter.

In some implementations, heart rate data can be computed over extended periods of time, and can be used to trigger data acquisition by the implantable device. In some cases, slope of the patient's trended heart rate may be used in the analysis method. For example, intervals where the patient's heart rate is accelerating, which may be indicated by an increasing slope of the trended heart rate data, may coincide with periods where an increased cardiac burden (e.g., an increased repolarization alternans burden) may indicate patient cardiac vulnerability.

Heart rate variability or changes in heart rate may serve as a surrogate for autonomic tone, according to some implementations. For example, heart rate variability can serve as an indicator of a patient's cardiac autonomic modulation. Because short-term heart rate regulation may be predominantly governed by sympathetic and parasympathetic neural activity, examining heart rate fluctuations can provide a window for observing the state and integrity of the autonomic nervous system. Long-range heart rate variability measures can provide information useful in prognostic prediction, and can include the standard deviation of the mean values of successive heart period epochs and power in very-low frequency (VLF) bands. Reductions in SDANN and VLF can indicate poor survival prospects for patients, for example, if they have chronic, severe mitral regurgitation, an acute or recent myocardial infarction, or idiopathic dilated cardiomyopathy, or have been assessed for arrhythmias, as described for example in Stefano Guzzetti et al., Different Spectral Components of 24 h Heart Rate Variability are Related to Different Modes of Death in Chronic Heart Failure, Eur. Heart J., (2005) 26: 357-62, and Serge Boveda et al., Prognostic Value of Heart Rate Variability in Time Domain Analysis in Congestive Heart Failure, J. Interventional Cardiac Electrophysiology (June 2001) 5(2): 181-87.

In some implementations, other power spectral density parameters of HRV data may be computed. For example, power spectral density may be separated into multiple frequency zones, such as very low (e.g., below about 0.04 Hz), low (between about 0.04 Hz and 0.15 Hz), and high (between about 0.15 Hz and 0.4 Hz), see A. Malliani et al., Cardiovascular Neural Regulation Explored in the Frequency Domain, Circulation, (1991) 84: 482-92. The high frequency band is believed to be dominated by the parasympathetic nervous system, while the low frequency band is believed to be mediated by sympathetic and parasympathetic nervous outflows, see id. LF-to-HF ratios may be used to access autonomic balance as an approximation. However, recent studies suggest that the parasympathetic contributions to LF may be as significant as those of the sympathetic nervous activities; consequently, the LF-to-HF ratio may not be an accurate measure of the autonomic balance. The principal dynamic mode can be used to separate dynamics of the two nervous systems, as described in Yuru Zhong et al., Quantifying Cardiac Sympathetic and Parasympathetic Nervous Activities Using Principal Dynamic Modes Analysis of Heart Rate Variability, Am. J. Physiology Heart Circ. Physiology, (September 2006) 291:H1475-83. It is based on extracting only the intrinsic dynamic components of the signal via eigendecomposition. See id.

Next, sensitivity of cardiac function to sympathetic drive is calculated at step 306. In some implementations, sensitivity of cardiac function can be calculated by computing a ratio of change in an alternans burden to a change in sympathetic marker tone. In other implementations, sensitivity of cardiac function can be calculated by computing a ratio of change in a hypertension burden, a QT interval, or one or more delayed afterdepolarizations to a change in sympathetic marker tone. Sympathetic tone markers and cardiac function indicators may be tracked, and the sensitivity of cardiac function may be calculated as an onset value of the sympathetic tone marker at which a sustained elevation in indicator of proarrhythmogenic substrate appear. In some implementations, the onset value may be expressed as a heart rate value, but it could similarly be expressed, for example, as a percentage of the patient's maximum heart rate, or as a range of heart rate values.

The calculated sensitivity of cardiac function to sympathetic drive can be stored, and a trend of sensitivities over time can be compiled. In an implementation, the trend may include a plot of sensitivity of cardiac function to sympathetic drive versus time. In another implementation, the trend may include a plot of slope of sensitivity of cardiac function to sympathetic drive versus time.

As shown in FIG. 4, sensitivity of cardiac function to sympathetic drive can by calculated by computing a ratio of change in burden to change in sympathetic tone marker (306a). Alternatively, sensitivity of cardiac function to sympathetic drive can be calculated by determining an onset value of sympathetic tone marker at which sustained elevation in the burden appears (306b). The sustained burden may be compared to a predetermined threshold level, for example, or alternatively the threshold may be updated over time based on changing circumstances. In some implementations, analysis and trend assessment may focus on short durations, such as data collected over the preceding several hours, one hour, forty-five minutes, thirty minutes, twenty minutes, fifteen minutes, ten minutes, or less. In this manner, an abrupt change in indication of proarrhythmogenic substrate, in sympathovagal balance, or in both, can be used to identify a near-term risk of a dangerous cardiovascular insult. When this occurs, a therapy that modulates sympathovagal balance can be initiated. In some cases, the system may direct that the therapy be initiated, with actual administration performed outside of the system.

In various implementations, the cardiac or cardiovascular signal may be measured at multiple periods in time, such as over several seconds, minutes, hours, days, weeks, months, or years, and each recording may include information corresponding to multiple cardiac cycles. Using the measured signal, sensitivity of cardiac function to sympathetic drive may be calculated for each of the periods, and the sensitivity may be trended over time. The trend of the sensitivity over time may be evaluated to determine an indicator of a degree of cardiac risk for the patient. In some implementations, changes in the trend over time may be used to establish a risk indicator value indicative of a patient's susceptibility to sudden cardiac death, for example. In some implementations, changes in the trend over time may be used to alert to disease worsening.

In various implementations, circadian variation or variability may be considered when evaluating trends of sensitivity over time. For example, with some implementations, the analysis can occur on a daily basis, such as corresponding to a particular time each day. The analysis can alternatively be performed over other time periods, such as on an hourly, weekly, or monthly basis. In some implementations, a trend may be adjusted to account for circadian variability. For example, a patient's sympathetic tone may vary substantially over the course of a day due to circadian rhythms that affect physical parameters within the body. Such impact may occur independent or semi-independent of patient activity levels, according to some implementations. By factoring circadian variability into cardiac risk assessment determinations, it may be possible to obtain more accurate results according to some implementations. In some implementations, in addition or in lieu of consideration of circadian variability over a single day, variability over two or more (three, four, five, etc.) days may be considered, and the trending information may be appropriately adjusted to account for the variability. Using the methods disclosed here, such changes may be unmasked and used to refine the analysis procedure to more accurately assess a patient's cardiac state, or assess therapy effectiveness for the patient. In other implementations, the signal may be filtered to average over circadian variability and produce a long-term trend. In some cases, for example, circadian variability can be used to adjust for variability in developing a baseline trend.

As described, the methods consider a relationship over time between a cardiac burden and a heart rate parameter, as opposed to looking at a snapshot in time. The relationship is tracked and trended over time, typically in an ambulatory setting where the patient is free to go about their daily activities without the inconvenience of arranging and visiting a clinical facility for dedicated testing during a condensed time period. As such, the results obtained using the methods disclosed here may provide more accurate assessment data in some implementations, and may be more convenient for the patient.

FIGS. 5A, 5B, and 5C are exemplary charts of data trended over time. The charts 5A, 5B, 5C share a common horizontal axis 505 of time listed by month, and display data covering an exemplary monitoring period from the beginning of February through the end of April, in this example. FIG. 5A illustrates an exemplary trend 510 in TWA amplitude over time, and shows T-wave alternans amplitudes 515 (the light gray signal) plotted against time, and a filtered TWA amplitude signal 520 (the black signal) superimposed over the TWA amplitude data 515. As will be described below, TWA provides one example of a cardiac burden, but any of the indicators of cardiac burden discussed herein may alternatively be trended. FIG. 5B illustrates an exemplary trend 525 in HR onset over time, and shows HR onset values 530 (the light gray signal) plotted against time, and a filtered HR onset signal 535 (the black signal) superimposed over the HR onset signal 530.

FIG. 5C trends cardiac risk 540 versus time. The cardiac risk trend 540 can be determined using data from the trended data shown in FIGS. 5A and 5B. The cardiac risk trend 540 may be determined in a variety of ways. As one example, an autoregressive model that uses a sliding window with time-varying coefficients can be used to trend the data. Changes in the coefficients can be added or summed to produce a weighted and bounded cumulative sum chart in the plot of FIG. 5C. The weights can be adjusted based on HR onset data, such as the data shown in FIG. 5B. The cumulative sum can be evaluated to detect persistent shifts in the trended signal data that may indicate changes in cardiac risk. In some implementations, evaluation to detect changes in the patient's symptoms indicated by shifts in the sensor data can involve comparing the cumulative sum to one or more thresholds, such as threshold 545. In the exemplary plot shown in FIG. 5C, an increase in cardiac risk, including an increase 560 above the threshold 545, is followed by two ventricular fibrillation episodes 550, 555. Using techniques described herein, an alert may be provided (e.g., to a health care professional or to the patient) when the cardiac risk trend meets or exceeds 560 the threshold 545, so that therapeutic interventions may be initiated or modified in an attempt to prevent sudden cardiac death, as may be caused by ventricular fibrillation episodes 550, 555.

The data shown in FIGS. 5A-5C covers a period of several months, and the analysis described with respect to FIGS. 5A-5C may be appropriate in monitoring longer term health trends for a patient. In detecting an imminent or near-term risk of a ventricular arrhythmia or an acute myocardial ischemia, either of which can result in SCD, it may be appropriate to consider a much shorter time period than considered in FIGS. 5A-5C. For example, analysis similar to that described with respect to FIGS. 5A-5C can be applied over a time window covering several hours (two, three, four, five, six, eight, twelve, sixteen, twenty-four, e.g.) or even periods less than one hour (e.g., forty-five, thirty, twenty, fifteen, ten, or five minutes). In this fashion, it may be possible to detect changes in proarrhythmogenic substrate indicators and sympathovagal balance that indicate a likelihood of a near-term cardiovascular insult.

In some implementations, trended data that exceeds a threshold may indicate that the patient's risk of an adverse cardiac event, such as sudden cardiac death, has advanced to a level where medical intervention is advisable. In these cases, a therapy the modulates sympathovagal balance, such as electroacupuncture or magnetoacupuncture, may be initiated, and optionally an alarm may be transmitted to the patient or an emergency responder.

FIG. 6 is a series of exemplary charts 600 that can be used to assess a patient's cardiac risk profile. As opposed to the longer timeframe considered with respect to FIGS. 5A-5C, FIG. 6 details a short time period of about sixteen minutes. A first chart 605 shows heart rate changes over a period of time by plotting heart rate versus time, using a vertical axis 606 of heart beats per minute and a horizontal axis 608 of minutes. Heart rate can be measured periodically over the given time interval, according to various implementations. A second chart 610 shows T-wave alternans amplitude over the same time period, using a vertical axis 611 of microvolts and a horizontal axis 613 of minutes. In other implementations, one or more different indicators of cardiac burden or proarrhythmogenic substrate, such as QRS alternans, mechanical alternans from a hemodynamic signal, a hypertension burden, QT interval changes, or delayed afterdepolarizations may be alternatively substituted or used in conjunction with the trend of TWA 610. As can be seen with reference to the first and second charts 605, 610, TWA generally have higher amplitude during periods of higher heart rates. Higher heart rates may indicate increased sympathetic tone, and the calcium ions that mediate the propagation of the heart's electrical signals may not have time to fully cycle at high heart rates, which can lead to alternans in the T-wave of the ECG.

A third chart 615 combines information from the first and second charts 605, 610, to display TWA amplitude versus heart rate. As such, the third chart provides information on the relationship between TWA and heart rate, so that the relationship may be trended over time, and changes in the trend may be tracked and correlated to patient progress or vulnerability. A vertical axis 616 has units of microvolts, while a horizontal axis 618 has units of heart beats per minute. Additionally, the plot uses line width to depict periods where the patient's heart rate is increasing (narrow line width 620) or decreasing (wider line width 625). The charts in the series 600 show data collected over a short time period, but in other implementations the data may be collected over one or more days, weeks, months, or years, and the data may be trended in similar fashion.

The third chart 615 provides an example of an indicator of cardiac function (the TWA in this case) versus a measure of sympathetic drive (heart rate in this case). A trend of the sensitivity of the cardiac function to sympathetic drive may be evaluated for risk stratification purposes, or to assess risk of an impending cardiac insult. In some implementations, an indicator of a degree of cardiac risk can be determined using the chart 615, such as by determining an onset value of a sympathetic tone marker.

The onset value may correspond to a sympathetic tone marker value at which a sustained elevation in the cardiac burden appears. In this case, a heart rate at which sustained increase in TWA amplitudes appear. For a given cardiac burden threshold, the onset value may be the lowest sympathetic drive measure at which sustained burden occurs, such that at sympathetic drives above the onset value, cardiac burden is measured above a threshold value for at least a predetermined period or percentage of time. T-wave alternans can be considered significant at specific voltage levels, and the voltage levels may be varied to account for numerous factors, according to some implementations. In some implementations, T-wave alternans can be considered significant above about 1.9 microvolts, although other threshold levels can be used in other implementations (e.g., about 3 microvolts in the FIG. 6 series of charts 600). For example, a first circled portion 630 may correspond to a first onset value of about 87 beats per minute for decreasing heart rate, and a second circled portion 635 may correspond to a second onset value of about 107 beats per minute for increasing heart rate in this example. In some implementations, a single onset value may be calculated that does not distinguish between periods of increasing and decreasing heart rate. In some cases, circadian variability factors can be used in determining appropriate thresholds, and the thresholds can be adjusted depending on time of day or other circadian factors.

It has been demonstrated that onset HR over which significant TWA occur is higher in healthy controls than in high-risk patients. For example, it has been demonstrated that that at higher heart rates, TWA becomes a more sensitive but less specific test for cardiac risk, see Neal Kavesh et al., Effect of Heart Rate on T wave Alternans, J. Cardiovascular Electrophysiology, (1998) 9:703-08.

The implantable device 102 can measure a cardiac signal and determine, using any of the methods discussed above, a degree of risk of near-term (e.g., with about less than 20 minutes) ventricular arrhythmia or acute myocardial ischemia (i.e., a sever cardiac insult) that may result in SCD. When such a risk is identified, the implantable device may signal an external therapy device to administer a therapy that modulates a sympathovagal balance, such as by activating parasympathetic drive, so that the patient's sympathovagal balance may revert to a benign level and thereby sufficiently reduce the patient's susceptibility to the cardiac insult so that the insult is averted. In some implementations, the analysis may be carried out, in whole or in part, by one or more external devices, such as the external therapy device 104, the external communications device 132, or the external computing device 146. As described above, alarms or warnings may optionally be transmitted to the patient or to an emergency responder.

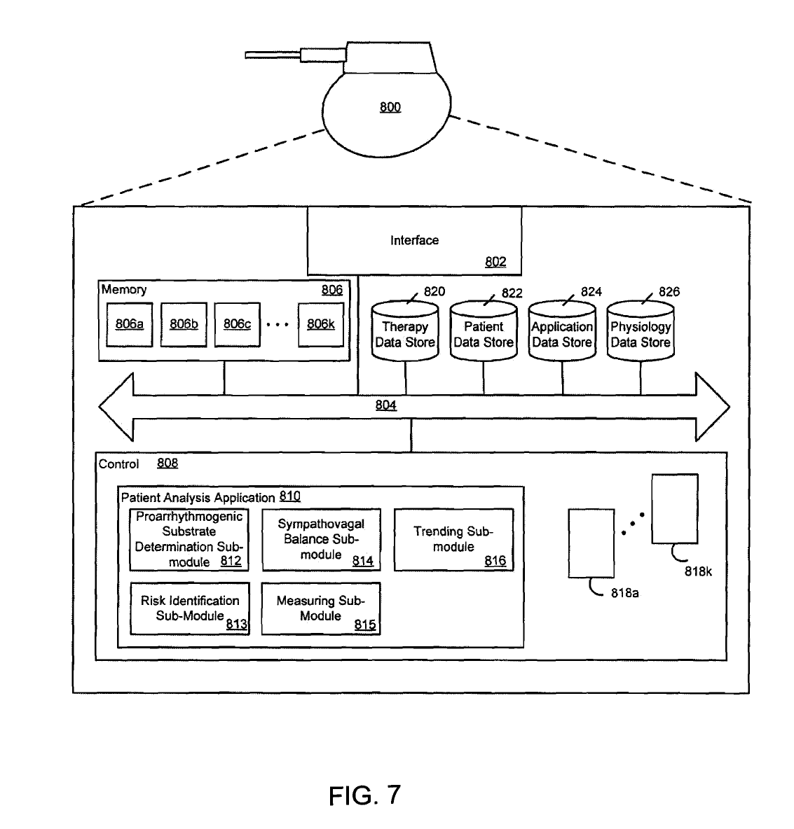

FIG. 7 is a block diagram of an exemplary device 800 that can be used to predict an upcoming cardiovascular insult, and taking measures to prevent its occurrence. In various implementations, the device 800 can implement all or of portion of the techniques disclosed herein. For example, the device 800 may correspond to the implantable device 102 shown in FIG. 1. In other implementations the device 800 may correspond to the external therapy device (104a or 104b), or to the external communication device 132 of FIG. 1C. In general, the device 800 receives cardiac signal data, determines a risk of impending cardiovascular insult, and administers or directs administration of an acupuncture therapy that modulates sympathovagal balance. In various implementations, the device 800 will include only a subset of the internal components pictured. Similarly, the device 800 may take different form factors, and may include various sensors or sense electrodes for measuring a cardiac or cardiovascular signal. For example, the device 800 may include one or more sense electrodes on an exterior surface of the device 800, or a sense port (e.g., a pressure sense port) on an exterior surface of the device 800. Also, the device 800 may include one or more leads (e.g., a subcutaneous lead or an intracardiac lead) or pressure sense catheters that may include various electrodes or sensors for measuring physiologic signals.

Cardiac signal data can be received by the device 800 through an interface 802. In implementations including external devices, the interface 802 may receive data over a communication channel or over a network, for example. In implementations implementing the techniques within implanted devices, the interface 802 may receive sensed signal readings, such as from connected electrodes or other types of sensors (e.g., a pressure sensor or blood flow sensor). In some implementations, the interface 802 includes a telemetry component that may be able to transmit or receive data wirelessly over an antenna (not shown in FIG. 7). The interface 802 can place the cardiac signal data on a bus 804, which provides interconnectivity between the various modules and components of the device 800. A control module 808 may include hardware and software modules, including one or more processors (not shown) that may execute instructions to perform tasks for the system, such as the steps comprising the methods disclosed herein. Examples of processors that may be suitable can include one or more microcontrollers, microprocessors, central processing units (CPUs), computational cores instantiated within a programmable device or ASIC, and the like. In general, the processor or other control components of the control module 808 may control or manage the flow of information throughout the system, including the flow of information over the bus 804. As is conventional, instructions and data may be stored in a non-volatile data store, and may be moved to a memory 806 for active use. In some implementations, the memory 806 can store the cardiac signal data within bins 806a through 806k. The processor may access instructions and data from memory for execution, for example, and may load the instructions and data into on-chip cache, if equipped and as appropriate.

The control module 808 includes a patient analysis application 810, which can be used to implement the risk detection and therapy direction techniques discussed herein. The patient analysis application 810 includes an proarrhythmogenic substrate determination sub-module 812, a sympathovagal balance sub-module 814, a risk identification sub-module 813, and a trending sub-module 816, each of which may implement portions of the techniques discussed herein. A measuring sub-module 815 may be used to control measurement of one or more cardiac or cardiovascular signals. The control module 808 can also optionally include other applications 818a through 818k, which may be used to perform other tasks associated with the device.

The control module 808 may request data from various data stores 820, 822, 824, 826, any or all of which may be optionally omitted in various implementations. For example, a therapy data store 820 may store data relating to patient therapies, a patient data store 822 may store patient-specific information, an application data store 824 may store information relating to graphically displaying trending information, and a physiology data store 826 may store medical information relating to possible medical conditions. The control module 808 can process cardiac signal data and pass the processed data or information derived from analysis of the data to the interface 802 over the bus 804. From there, the interface 802 may forward the data, for example, to a monitoring device for review by a health care provider.

The control module 808 can execute instructions that cause the module to implement the techniques discussed above. In some implementations, the proarrhythmogenic substrate determination sub-module 812 can determine a cardiac burden (e.g., an alternans burden or a hypertension burden), QT interval change information, or delayed afterdepolarization information from a cardiac signal. For example, if the signal is an ECG signal, T-wave alternans information can be determined. In some implementations, the proarrhythmogenic substrate determination sub-module 812 can use harmonic decomposition to analyze the cardiac signal using efficient, time-frequency analysis techniques. Examples of techniques that can be used to determine alternans information from a cardiac or cardiovascular signal can be found in U.S. Provisional Application No. 60/991,650, referred to previously above. In other implementations, the proarrhythmogenic substrate determination sub-module 812 can use time domain analysis to determine alternans information. In still other implementations, frequency domain analysis can be used to determine alternans information. Similarly, a hypertension burden can be determined from the cardiac signal. The hypertension burden may be due to elevated systolic pressure, elevated diastolic pressure, or a combination of both.

In some implementations, the sympathovagal balance sub-module 814 can determine various autonomic parameter information from the cardiac signal data. In some implementations, the sympathovagal balance sub-module 814 can determine heart rate information, such as heart rate variability information or heart rate change information. Other autonomic parameters that can be determined, with some implementations, include heart rate turbulence and/or deceleration capacity. In some implementations, indicators of sympathetic drive may be determined from the cardiac signal. Heart rate may be determined, and heart rate changes may be tracked, including distinguishing between periods of increasing heart rate and periods of decreasing heart rate. Periods may be further distinguished based on heart rate rate-of-change, or slope. In various implementations, cardiac signal data, or information derived from the data, may be stored according to these distinctions, which may facilitate improved patient analysis.

The trending sub-module 816 may calculate a sensitivity of cardiac function to sympathetic drive for data corresponding to multiple cardiac strips measured at multiple periods of time, such as over multiple minutes, hours, days, weeks, or years, although for risk assessment of cardiac episodes that can result in sudden cardiac death, shorter-term periods of analysis covering several minutes or tens of minutes, up to an hour or two may be appropriate. These calculations may use information from the proarrhythmogenic substrate determination sub-module 812 and the sympathovagal balance sub-module 814. Trending steps may include trending an alternans burden (TWA, QRS, or mechanical alternans, e.g.), hypertension burden, QT interval data, delayed afterdepolarization data, heart rate, heart rate slope, HRV, HRT, deceleration capacity, or relationships among two or more of the foregoing. The risk identification sub-module 813 can then evaluate a trend of the sensitivity over time as an indicator of a degree of cardiac risk for the patient, including assigning a risk value according to how likely the patient is to suffer a dangerous cardiac insult within a predetermined period of time. In some cases, circadian variability factors can be used to adjust the trended information, or used to adjust risk identification results in view of the trended information. The circadian variability analysis may consider that at certain times of the day, sympathetic drive may tend to be higher than at other times of the day, independent of present patient activity.

In some implementations, one or more of other applications 818a through 818k can compile a display of the trending information. In some implementations, the control module 808 can receive information from other sources and apply the information to calculations in the patient analysis application 810.

Information applied to calculations determined in the patient analysis application 810 can include data from various data stores within the device 800. Example data stores can include the therapy data store 820, the patient data store 822, the application data store 824, and the physiology data store 826. These data stores can store information that can be used in assessing a patient's cardiac risk profile, therapy effectiveness, or patient compliance with a therapy regimen. In some cases, the information may be received from a health care professional, for example. The data stores can also store information received from a patient. The data stores can be updated on a periodic basis. In the depicted implementation, the data stores 820-826 reside within the device 800, but in other implementations one or more of the data stores, or other data stores storing other relevant information, may be external to the device, and may be accessed by the device 800 or by another device that provides the information to the device 800.

The therapy data store 820 can store information regarding patient therapy methods, such a drug pump, electroacupuncture or magnetoacupuncture therapy to induce parasympathetic activation, a cardiac rhythm management device, topical medication applicators (e.g., a patch to release a topical anaesthesia), or oral medications. For example, information on each of the cardiac acupoints described above may be contained in the therapy data store 820. In some implementations, varying amounts of acupuncture therapy may be administered depending on the particular acupoint targeted.

Information regarding the patient can also aid in determining patient cardiac risk. The patient data store 822 can include patient information such as drug allergies, previous cardiac history, and current or historical health care providers. The control module 808 can use information from the patient data store 822 to modify an assessment of trending information. The application data store 824 can be used to store information for any of the applications that may run on the device 800, of that may be used for communicating with other devices. For example, the application data store 824 may contain libraries of information that various applications may use in operation.

The physiology data store 826 can also provide information to aid in determining cardiac state by including the patient's physical data. The physiology data store 826 can include patient vital signs from previous visits, or other risk markers determined with external or implantable devices, such as a burden of non-sustained ventricular tachycardia, and other pre-existing conditions like genetic predisposition to cardiac disease, to list just a few examples. This data can be used with the techniques described here to provide a more accurate risk assessment, according to some implementations.

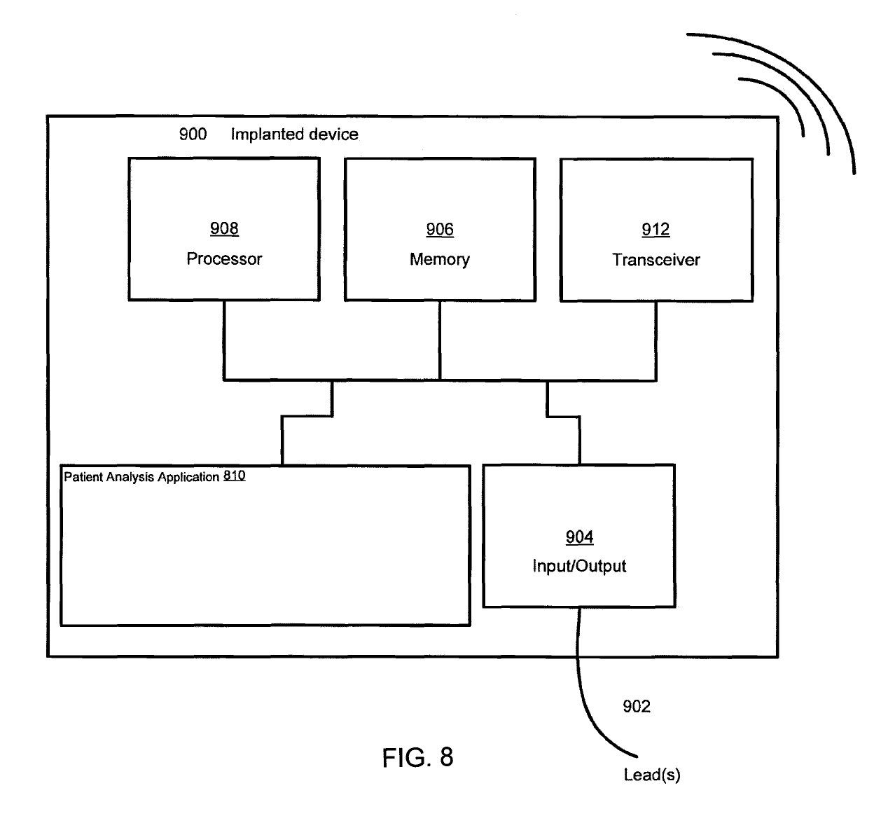

FIG. 8 is a simplified block diagram of an exemplary implantable device 900. In some implementations, the device 900 can implement any of the methods described herein, or any portion of the methods. In some cases, the device 900 can cooperate with one or more other devices (whether implanted or external) to implement the methods discussed herein. While not shown here for simplicity, in general the device 900 may include some or all of the components discussed above with reference to the device 800 of FIG. 7. In some implementations, the device 900 can implement a portion of some of the methods described herein (e.g., the device may measure and sense a cardiac or cardiovascular signal, store the signal, and wirelessly transmit the signal or a portion of the signal to an external device for further processing, without performing cardiac burden analysis, sympathetic drive analysis, trending analysis, or risk assessment). Implementations of the device 900 can be used to accurately measure risk indicator assessment over a period of time. In general, the implanted device 900 records and processes a patient's cardiac signal data. For example, the implanted device 900 can record an ECG signal, an impedance signal, or a hemodynamic signal. In some implementations, the device may sense and record multiple cardiac or cardiovascular signals, such as both an electrical signal and a hemodynamic signal, either or both of which may be analyzed (independently or cooperatively) with various autonomic parameters to determine cardiac risk.

The depicted device 900 includes one or more leads 902, which may be configured for positioning inside or outside of a patient's heart or other bodily organ, depending on the implementation, an input/output module 904, a memory 906, a processor 908, a transceiver 912, and the patient analysis application 810, described above with reference to FIG. 7, pictured stored in a non-volatile memory medium. The transceiver 912 may include a transmitter and a receiver, and may communicate wirelessly with an external (or implanted) device using an antenna (not shown). For example, the implanted device 900 may communicate with the external therapy device 104 in FIG. 1, or with an intermediary external communication device as described above. In some implementations, the implanted device 900 can incorporate the transceiver 912 and the input/output module 904 into the same module. In some implementations, the transceiver 912 can be configured to receive command signals. For example, the receiver 912 can receive a command that instructs the device 900 to record a segment of cardiac signal data.

The one or more leads 902 can include one or more electrodes that can sense signal data, including cardiac signal data of the patient. In some implementations, the one or more leads 902 are intracardiac leads; in some implementations, the one or more leads 902 are configured for subcutaneous positioning within a patient; in some implementations, at least one intracardiac lead and at least one subcutaneous lead are included. In some implementations, the one or more leads 902 may be replaced or supplemented with one or more sensors or ports configured to sense a hemodynamic signal. Some implementations may include one or more catheters that may facilitate hemodynamic measurements at a distance from the device. For example, a pressure transmission catheter may be used to sense a body pressure and refer the pressure to a pressure transducer, which may be housed within the body of the device 900 or in a separate housing, in which case the pressure information may be communicated to the device 900 by wired or wireless communication link. Various combinations of leads and electrodes are possible. As one example, the device may include a single lead with a single electrode, and may include a second electrode on the housing. As another example, a lead may include two or more electrodes, or the housing may include two or more electrodes. Leadless implantable devices are also contemplated, where an exterior surface or the device includes electrodes and/or sensor(s) to make the measurements discussed herein.

In general, the processor 908 can execute instructions to perform the methods described herein. For example, the patient analysis application 810, and its various sub-modules, may include instructions that when executed perform one or more of the methods discussed herein. The patient analysis application 810 may be stored in a non-volatile medium (e.g., EPROM, flash memory, EEPROM, or various other non-volatile storage mediums familiar to those skilled in the art) within the device 900, and may be transferred to memory 906 (e.g., SRAM, DRAM, SDRAM, or various other volatile or non-volatile storage mediums familiar to those skilled in the art) for active use by the processor 908. Additional device components, such as a battery, signal processing circuitry, clocking circuitry, and optionally any of the components depicted in FIG. 7 are omitted from FIG. 8 for simplicity. In some implementations, the memory 906 and processor 908 may be implemented in a programmable device, such as a programmable logical device (PLD, e.g. an FPGA) or application specific integrated circuit (ASIC). In some applications, the patient analysis application 810 may include only a subset of the depicted sub-modules. In these implementations, for example, processing of the cardiac signal data may occur outside of the implantable device 900, such as in the external therapy device 104 shown in FIG. 1, or in any of the other devices described above.

Cardiac signal data may be recorded over a predetermined number of heartbeats, or for a predetermined time interval. Each recording of cardiac signal data may be referred to as a “strip” of data. In some implementations, the strips can contain data associated with 128 heartbeats, but strip lengths of any appropriate number of beats (e.g., 32, 64, 256, 512, etc.) or time period can be used. In some implementations, the implanted device 900 can make determinations using the cardiac signal data to decide whether to continue recording.

A number of implementations of the invention have been described. Nevertheless, it will be understood that various modifications may be made without departing from the spirit and scope of the methods, systems, and devices described herein. For example, the sensing device may be a stationary device, according to some implementations. The sensing device may be used in implementations where data is collected in a health care facility, such as a hospital. The sensing device can transmit data to be analyzed using the techniques disclosed herein. In implementations that use an implantable device, the implanted device may comprise two or more implantable enclosures. In some implementations, a risk indicator value derived from an electrical signal may be used in combination with a risk indicator value derived from a hemodynamic signal to assess a patient's cardiac state, or a single risk indicator value may be computed using both an electrical cardiac signal and a hemodynamic cardiac signal. Accordingly, other embodiments are within the scope of the following claims.

What is claimed is:

1. A method of providing therapy to a patient to prevent an occurrence of a dangerous cardiac event, the method comprising:
receiving a cardiac signal sensed over multiple cardiac cycles;
determining, using the received cardiac signal, a risk of impending cardiovascular insult by assessing an indicator of proarrhythmogenic substrate and a change in sympathovagal balance; and
administering, based on the determined risk, a therapy comprising acupuncture to modulate sympathovagal balance.

2. The method of claim 1, further comprising detecting, using the received cardiac signal, a cardiac instability trigger for use in determining the risk of cardiovascular insult.

3. The method of claim 2, wherein the cardiac instability trigger is one or more ectopic beats.

4. The method of claim 1, wherein the indicator of proarrhythmogenic substrate is an alternans burden that exceeds a predetermined threshold.

5. The method of claim 4, wherein in the alternans burden is a repolarization alternans burden determined from the cardiac signal.

6. The method of claim 4, wherein in the alternans burden is a QRS alternans burden determined from the cardiac signal.

7. The method of claim 4, wherein in the alternans burden is a mechanical alternans burden determined from the cardiac signal.

8. The method of claim 1, wherein the indicator of proarrhythmogenic substrate is a hypertension burden determined from the cardiac signal.

9. The method of claim 1, wherein the indicator of proarrhythmogenic substrate is one or more delayed afterdepolarizations, determined from the cardiac signal, that exceed a predetermined threshold.

10. The method of claim 1, wherein the indicator of proarrhythmogenic substrate is a QT interval change that exceeds a predetermined threshold.

11. The method of claim 1, wherein the change in sympathovagal balance is estimated from a heart rate variability measurement determined from the cardiac signal.

12. The method of claim 1, wherein the change in sympathovagal balance is estimated from a heart rate increase measurement determined from the cardiac signal.

13. The method of claim 1, wherein the therapy modulates sympathovagal balance by activating parasympathetic drive.

14. The method of claim 1, wherein the acupuncture comprises electro-acupuncture applied to one or more cardiovascular acupoints.

15. The method of claim 1, wherein the acupuncture comprises magneto-acupuncture applied to one or more cardiovascular acupoints.

16. The method of claim 1, wherein the cardiac signal is an electrocardiogram signal.

17. The method of claim 1, wherein the cardiac signal is a blood pressure signal.

18. The method of claim 1, wherein the impending cardiovascular insult is arrhythmia.

19. The method of claim 1, wherein the impending cardiovascular insult is acute myocardial ischemia.

20. The method of claim 1, further comprising initiating an alarm to alert an emergency responder to the impending cardiovascular insult.

21. The method of claim 1, further comprising issuing an alert in a manner that allows a preventative action to be taken to prevent the dangerous cardiac event.

22. The method of claim 21, wherein the preventative action comprises donning a wearable external defibrillator, and wherein the patient performs the action in response to the alert.

23. A system for providing therapy to prevent an occurrence of a dangerous cardiac event, comprising:
a monitoring component that receives a cardiac signal sensed over multiple cardiac cycles;
a processing component that determines, using the received cardiac signal, a risk of impending cardiovascular insult by assessing an indicator of proarrhythmogenic substrate and a change in sympathovagal balance; and
a therapy component that administers, based on the determined risk a therapy comprising acupuncture to modulate sympathovagal balance.
